(19) United States
(12) Patent Application Publication
(10) Pub. No.: US 2003/0165450 A1
(43) Pub. Date: Sep. 4, 2003

(54) Cosmetic composition forming a rigid coat, comprising a polymer with a non-silicone skeleton and containing reactive functional groups

(76) Inventors: Henri Samain, Bievres (FR); Isabelle Rollat-Corvol, Paris (FR); Franck Giroud, Clichy (FR); Nathalie Mougin, Paris (FR); Aude Livoreil, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/321,361

(22) Filed: Dec. 18, 2002

(30) Foreign Application Priority Data
Dec. 18, 2001 (FR) 01 16384

(51) Int. Cl. A61K 7/06; A61K 7/11

(52) U.S. Cl. 424/70.11; 424/70.17

(57) ABSTRACT

Cosmetic compositions, such as hair compositions, comprising at least one polymer with a non-silicone skeleton, comprising at least two reactive chemical functional groups, which are capable of forming a rigid coat on the hair, a cosmetic process comprising the application of this composition onto the hair, and its use for producing a rigid coat on the hair.

Disclosed herein are non-tackifying cosmetic compositions, such as hair compositions, comprising at least one polymer with a non-silicone skeleton, comprising reactive chemical functional groups, wherein the cosmetic compositions are capable of forming a rigid coat on the hair. Also disclosed herein is a cosmetic process for applying this non-tackifying composition onto the hair, as well as its use for producing a rigid coat on the hair.

To determine whether the free organic functional groups (F) of a polymer (P) constitute reactive chemical functional groups, test 1 described below may be performed:

(1) a solution or a dispersion of the polymer (P) in a cosmetically acceptable solvent chosen from water, C₁ to C₄ alcohols, esters and ketones, and such as water, is prepared, such solution or dispersion having a relative polymer content ranging from 0.1% to 50% by weight, relative to the total weight of the composition;

(2) for a period ranging from 1 to 60 minutes, the solution or dispersion of polymer (P) is left to stand or is subjected to at least one of the operations chosen from:

(i) it is stirred;

(ii) it is activated by a temperature ranging from 0° C. to 100° C.;

(iii) it is activated by a pH ranging from 1 to 13;

(iv) it is activated by at least one chemical additive (A) chosen from molecules and polymers bearing free chemical functional groups capable of reacting with at least one free organic functional group (F) of the polymer (P), wherein the chemical additive (A) can be, for example, a polymer bearing chemical functional groups identical to those of the hair, wherein the chemical functional groups are chosen from amine, alcohol, carboxylic acids, disulphide and thiol functional groups;

(3) the solution or dispersion of polymers (P) is examined by methods known to those skilled in the art, such as by infrared or RAMAN spectroscopy, in order to determine if at least one free organic functional group (F) of the polymer (P) has given rise to the formation of covalent bonds, which link, for example:

two atoms present in free organic functional groups (F) belonging to different polymers (P), and

one atom present in the polymer (P) and one atom present in the chemical additive (A);

(4) the polymer (P) is termed a “polymer comprising reactive functional groups” if the formation of covalent bond(s) is detected in point (3) and provided that such a covalent bond does not result exclusively from a hydrolysis or an oxidation of the polymer.

As disclosed herein, the expression “polymer with a non-silicone skeleton” means a polymer not consisting exclusively of —Si—O—Si— sequences in its main chain.

As disclosed herein, the term “coat” means an envelope formed at the surface of each hair, after drying the non-tackifying cosmetic composition. This envelope has virtually the shape of a hollow cylinder which may extend from the root to the end of the hairs and which adheres strongly thereto.

The at least one polymer with a non-silicone skeleton comprising reactive functional groups as disclosed herein, is capable of forming covalent bonds by carrying out the test 1 described above. This characteristic distinguishes the at least one polymer disclosed herein from most of the polymers known in the field of hair compositions, which do not react, under the conditions of the test 1, by forming strong bonds, but at the very most by interacting with each other or with additives via bonds of hydrogen bonding or saline type.

The at least one polymer disclosed herein excludes polymers with a non-silicone skeleton containing photo-activatable reactive functional groups, i.e. polymers comprising chemical functional groups which, when irradiated at a wavelength ranging from 200 to 800 nm, give rise, in at least one step, to the formation of new covalent bonds.

Cosmetic products intended for hair treatment often use polymers. They make it possible to obtain, for example, hairstyle holding effects, softness effects or sheen effects. Some compositions using polymers can have drawbacks that may be inconvenient. For example, if, after a product containing polymers has been applied, a person passes his hand through his hair, some of the polymers may be deposited on his fingers during the contact. This transfer phenomenon, even if only partial, can leave an impression of dirty or sticky hair. The extent of this transfer may depend on the climatic conditions. Thus, it is often, for example, pronounced in a humid environment.

Moreover, when the hair is covered with sebum, either along its length or at the root, and a cosmetic product is applied thereto, for instance a styling product, the said product may not only be ineffective, but, in addition, may make the hair even more artificially shiny and dirtier. Another drawback of the polymers used in cosmetics lies in the fact that they occasionally dry out the hair, thus can cause an impairment in its feel and a reduction in the expected effect of the product, for example, the fixing and/or hairstyle holding effect. An additional drawback that may also be mentioned is the fact that the polymers deposited on the hair can be very quickly removed during shampooing. For example, often the polymers used to form a coat on the hair, such as a film of rigid consistency, can result in the feel of the hair being, for example, coarse or sticky, and unpleasant. In addition, this rigid coat can be removed immediately upon washing the hair, and it is thus necessary to reapply the product, at least after each shampoo wash.

There is thus a need to produce non-tackifying cosmetic compositions that are improved with respect to the compositions of the prior art, and, for example, that do not stick to the fingers after application to the hair, do not dry out the hair, and can give the hair at least one good cosmetic property, even in the presence of sebum, and can be remanent with respect to repeated washings.

The present inventors have discovered, surprisingly and unexpectedly, that it is possible to achieve at least one of the objectives listed above by selecting the polymers introduced into non-tackifying cosmetic compositions on the basis of the nature of the chemical functional groups they bear and on the basis of the characteristics of the film they form on the hair.

Thus, disclosed herein is a non-tackifying cosmetic composition, such as a hair composition, comprising, in a cosmetically acceptable medium, at least one polymer with a non-silicone skeleton, comprising at least two non-photo-activatable reactive chemical functional groups, which may be identical or different, characterized in that:

(i) a film obtained by drying the composition at room temperature (22±2° C.) and at a relative humidity of 50%±5% has a Young's modulus ranging from 100 to 2000 MPa, measured at a thickness of 0.5 mm and at a traction of 20 mm/min,

(ii) the at least two non-photo-activatable reactive chemical functional groups are chosen from the following monovalent and divalent groups:

epoxy,

anhydride,

acid chloride,

ethyleneimino,

aldehyde,

acetal and hemiacetal,

aminal and hemiaminal,

ketone, α-halo ketone and α-hydroxy ketone,

lactone and thiolactone,

isocyanate,

thiocyanate,

N-hydroxysuccinimide ester,

imide,

imine,

imidate,

oxazoline, oxazolinium, oxazine, and oxazinium,

pyridylthiol,

thiosulphate,

acetoalkylate corresponding to the formula:

—OCO—A′—COCH₃,

wherein A′ is chosen from a bond, and linear and branched alkylene groups comprising from 1 to 5 carbon atoms,

AX, and

ASO₂X,

wherein A is chosen from alkylene, arylene and aralkylene groups comprising from 1 to 22 carbon atoms, which may be optionally interrupted with at least one unsaturated ring, and may optionally comprise at least one hetero atom, such as N, S and O,

X is a leaving group chosen from halogens, OSO₃H, OSO₂CH₃, OSO₂C₂H₅, OSO₂Tos, N(CH₃)₃, OPO₃R₂, and CN, and wherein Tos represents a tosylate group, and R is chosen from a hydrogen atom and C₁ to C₅ alkyl radicals; and

(iii) the at least one polymer with a non-silicone skeleton comprising at least two reactive chemical functional groups is other than vinylbutyral/vinyl alcohol/vinyl acetate copolymer.

“Tackifying compositions” are compositions that provide, after application to keratin fibers and drying, a styling material that has a delamination profile defined by at least:

a maximum delamination force Fₘₐₓ>1 newton, and

for example, also, a separation energy E_{s(M/V)} of the material placed in contact with a glass surface, of less than 300 μJ,

wherein Fₘₐₓ and E_{s(M/V)} being measured according to the protocol described in international patent application WO 98/38969, are excluded from the present patent application.

Another embodiment disclosed herein relates to a cosmetic method comprising the application of this non-tackifying composition to, for example, hair.

Yet another embodiment relates to the use of this non-tackifying composition to produce a rigid coat on the hair.

Without wishing to be bound by any theory, the inventors believe that the polymers with a non-silicone skeleton present in the cosmetic compositions as disclosed herein can, on account of their identical or different reactive chemical functional groups, react totally or partially either with themselves, with each other, with the hair, which may or may not be sensitized, and with at least one reactive constituent of the hair composition, and may do so after application of the cosmetic composition to the hair, to form a coat. The mechanism of formation of the coat may be understood more clearly by means of the following reaction scheme examples:

1) reaction of two polymers, each comprising two epoxy reactive functional groups with a reactive ingredient of the composition having the formula RHN—A—NHR′,

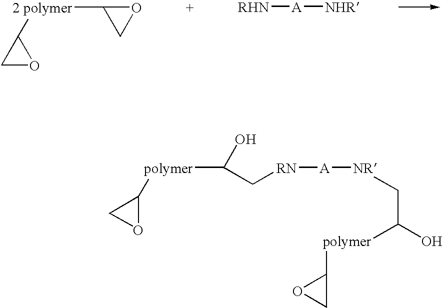

2) reaction of a polymer comprising two epoxy reactive functional groups with an amine functional group of the hair,

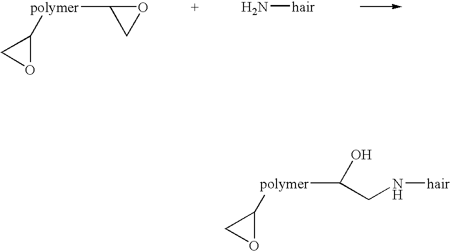

As described herein, the reaction of the polymers with a non-silicone skeleton with each other and with the hair may be promoted by supplying heat or by adding constituents, for example, pH regulators and chemical active agents such as oxidizing agents, reducing agents, inhibitors and polymerization catalysts.

In one embodiment, the polymer with a non-silicone skeleton comprising at least two reactive functional groups, comprises less than 50%, in numeric terms, of carboxylic acid ester functional groups relative to the total number of reactive chemical functional groups.

In another embodiment, the leaving group X is a halogen chosen from bromine, chlorine, iodine and fluorine.

When the polymer with a non-silicone skeleton, comprising at least two reactive functional groups, comprises at least one epoxy group, the epoxy group is, for example, monovalent and is chosen from groups corresponding to formula (I):

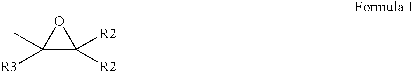

wherein R1, R2 and R3, which may be identical or different, are each chosen from:

a hydrogen atom;

linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;

aryl groups comprising from 6 to 22 carbon atoms;

aralkyl groups, wherein the alkyl group comprises from 1 to 20 carbon atoms; and

5- to 7-membered heterocycles.

When the polymer with a non-silicone skeleton, comprising at least two reactive functional groups, comprises at least one carboxylic acid anhydride group, the at least one carboxylic acid anhydride group is, for example, monovalent and is chosen from:

(a) groups corresponding to formula (II):

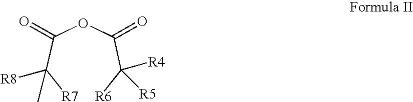

wherein, R4, R5, R6, R7 and R8, which may be identical or different, have the same meanings as those given for R₁, R₂, and R₃ in formula (I); and

(b) groups corresponding to formula (III):

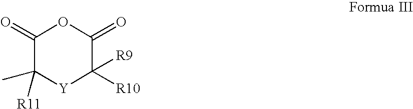

wherein Y is chosen from:

a bond;

hetero atoms chosen from O, N, S, Si and F;

alkyl and alkylene radicals that can be unsubstituted or substituted with at least one radical chosen from hydroxyl and amino radicals, comprising from 1 to 5 carbon atoms;

aralkylene radicals comprising from 7 to 10 carbon atoms;

polydimethylsiloxane radicals comprising from 1 to 6 silicon atoms; and

R9, R10, and R11, which may be identical or different, have the same meanings as those given for R1, R2, and R3 in formula (I).

When the polymer with a non-silicone skeleton, comprising at least two reactive functional groups, comprises at least one acetoalkylate group, the acetoalkylate group is, for example, included in a group corresponding to formula (IV):

—R′₁—OCO—A′—COCH₃ Formula IV

wherein R′₁ is obtained by eliminating a hydrogen atom of the radical R1 as defined in formula (I) and wherein A′ has the meaning given above.

When the polymer with a non-silicone skeleton, comprising at least two reactive functional groups, comprises at least one acid chloride group, the acid chloride group is, for example, included in a group corresponding to formula (V):

—R′₁—COCl Formula V

wherein R′₁ has the same meaning as defined in formula (IV).

When the polymer with a non-silicone skeleton, comprising at least two reactive functional groups, comprises at least one isocyanate group, the isocyanate group is, for example, is included in a group corresponding to formula (VI):

—R′₁—NCO Formula VI

wherein R′₁ has the same meaning as defined in formula (IV).

When the polymer with a non-silicone skeleton, comprising at least two reactive functional groups, comprises at least one acetal group, the acetal group is, for example, monovalent and included in at least one of the formulae (VII), (VIII) and (IX):

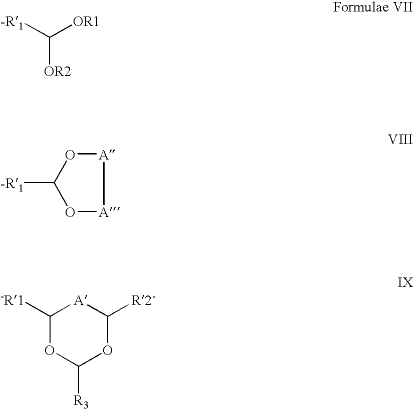

wherein:

R1, R2 and R3, which may be identical or different, have the same meanings as defined in formula (I).

R′₁ and R′₂ are obtained by eliminating a hydrogen atom of the radical R1 or R2 as defined in formula (I).

A′ has the same meaning as defined above.

A″ and A′″, which are identical or different, are each chosen from linear and branched alkyl and alkylene groups comprising from 1 to 5 carbon atoms, which may be optionally interrupted with at least one hetero atom chosen from O, N, S, Si and F, and may be optionally substituted with at least one radical chosen from hydroxyl and amino radicals.

In one embodiment, the at least one polymer with a non-silicone skeleton, comprising at least two reactive functional groups, is chosen from:

(a) copolymers synthesized from (meth)acrylate and acrylate monomers, comprising acetoacetate functional groups, corresponding to the general formula (IV.1):

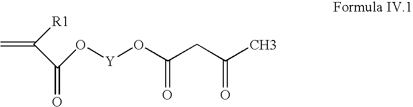

wherein R1 is chosen from H and CH₃, and

Y has the same meaning as defined in formula (III). In another embodiment, R1 is CH₃, and Y is —(CH₂)₂—.

(b) polymers synthesized from (meth)acrylate and (meth)acrylamide monomers comprising acetal functional groups, such monomers chosen from those of formulae (VII.1) and (VIII.1):

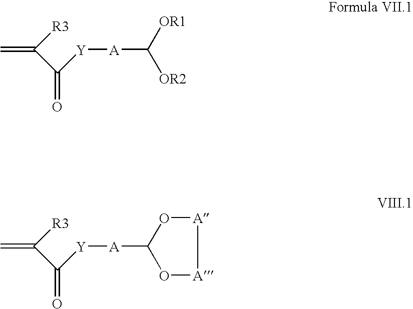

wherein Y is chosen from O and NH;

R3 is chosen from H and CH₃;

and A, A′, A′″, R1 and R2 have the same meanings as defined above.

Copolymers synthesized from N-ethyl acetal acrylamide of formula (VII.2)

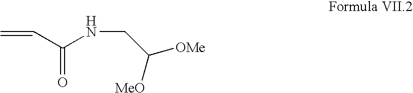

can also be used.

(c) copolymers comprising acetal functional groups, obtained by chemical modification of natural or synthetic polymers, such as the copolymers derived from the reaction of at least one aldehyde with poly(vinyl alcohol/vinyl acetate) of general formula (X):

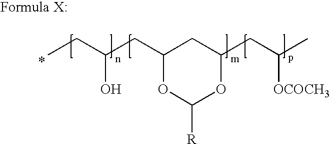

wherein R has the same meaning as defined above,

and n, m and p, which may be identical or different, each range from 1 to 10 000.

Such syntheses are known to those skilled in the art and are described in le Précis de Matières Plastiques, J. P. Trotigon, J. Verdu, Editions Nathan, 1996.

The polymers with a non-silicone skeleton used as disclosed herein may be obtained according to standard processes for polymerizing or modifying polymers.

To obtain such polymers, the production process may comprise, for example, at least one of the following operations:

a polycondensation,

an opening of at least one ring chosen from rings comprising from 2 to 9 carbon atoms and rings comprising from 2 to 4 silicon atoms, wherein the at least one ring may comprise at least one hetero atom, for example, N, O, S and Si; and

a polymerization of unsaturated monomers, chosen from free-radical and ionic polymerizations, by group transfer.

As disclosed herein, the polymer skeletons may be linear, branched, hyperbranched or dendritic. They may comprise at least one type of repeating unit, and thus may be homopolymers or copolymers which may be random, alternating, or block.

As disclosed herein, the reactive chemical functional groups are distributed along the main or side chains of the polymers, and may be optionally at the ends of the chains in the case of branched, hyperbranched and dendritic polymers.

When the polymer with a non-silicone skeleton as disclosed herein is formed by a polymerization process as described above, the reactive chemical functional groups may be present on the monomers serving as starting material for the polymerization, or may be formed by reaction of monomers with one another during polymerization, or may be provided by at least one chemical operation in addition to the polymerization, for example, an operation comprising grafting, such as onto the polymer obtained, molecular or polymeric units bearing appropriate chemical reactive functional groups chosen from those of formulae (I) to (IX).

To perform a polycondensation, the operating protocols described in “Step Polymerisation” in Principles of Polymerization, G. ODIAN, 3rd ed., Wiley Interscience, may, for example, be followed.

In the case of a polycondensation, the monomers used as the starting material are, for example, chosen from diamines and diols in reaction with diisocyanates, diacids, and diesters, which lead to polyurethanes, polyamides, polyesters and aziridines and derivatives thereof leading to polyalkyleneimines, such as polyethyleneimines and derivatives thereof.

For example, a polyurethane may be obtained by reacting the following monomers: isophorone diisocyanate, hexamethylene diisocyanate, methylenebis-(cyclohexane diisocyanate), and polytetramethylene glycol dihydroxyl.

To perform an opening of a ring chosen from rings comprising from 2 to 9 carbon atoms and rings comprising from 2 to 4 silicon atoms, wherein the at least one ring optionally comprises at least one hetero atom chosen from N, O, S and Si, the procedures described in “Ring Opening Ppolymerization” in Comprehensive Polymer Science, Perg. Press, vol. 3, may, for example, be followed.

In the case of a ring opening, the monomers used as the starting material to form the polymers are, for example, chosen from cyclic esters (lactones) and cyclic amides (lactams), such as:

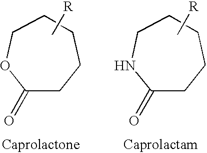

wherein R has the same meaning as defined as above.

When the polymer as described herein is formed by a production process comprising a ring-opening operation, the reactive chemical functional groups may be present in the monomers serving as the starting material and comprising a ring, for example, as chemical substituents present on the rings, may be formed after the mutual reaction of these monomers comprising a ring, may be provided by at least one chemical operation in addition to the ring-opening operation, for example, a separate operation comprising grafting molecular or polymeric units bearing the appropriate reactive chemical functional groups chosen from those of formulae (I) to (IX).

To perform a free-radical or anionic polymerization, the procedures described in “Radical polymerization and anionic polymerisation”, in Principles of Polymerization, G. ODIAN 3rd ed., Wiley Interscience, may, for example, be followed.

In the case of a free-radical or anionic polymerization, the monomers used as starting material to form the polymers are, for example, chosen from vinyls, dienes, (meth)acrylates and (meth)acrylamides.

In the case of a free-radical or anionic polymerization, the polymer comprises, for example, at least ten units linked via covalent bonds. The reactive chemical functional groups present on the polymer forming part of the compositions as described herein may be already present on the monomers serving as starting material for the free-radical reaction, may be formed during the free-radical reaction, or alternatively, for example, may be provided on the polymer by any additional chemical operation.

It is also possible to use natural polymers with a non-silicone skeleton and natural polymers that are chemically modified to include the reactive chemical functional groups listed above. Mention will be made, for example, and in a non-limiting manner, of polysaccharides (cellulose, chitosan, guar and derivatives thereof) and polypeptides (polyaspartic acid, polylysine and derivatives thereof). As described herein, such polymers with a non-silicone skeleton comprise, naturally or after modification, chemical functional groups chosen from hydroxyl, amine, carboxylic acid, thiol, aldehyde and epoxy functions, the reactivity of which is used without further modification in the composition (for example, with polymers bearing epoxy functions) or to provide the chemical functional groups listed above.

By way of non-limiting example, the polymer may be modified as follows:

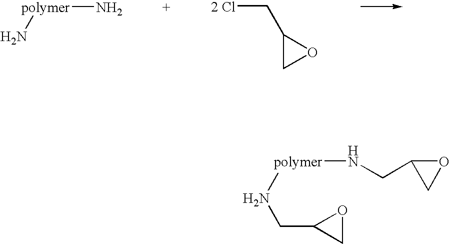

In order to determine the Young's modulus, a composition comprising the at least one polymer as disclosed herein is used in an amount that is suitable to obtain, in a Teflon matrix, a dry film 500±50 μm thick. Drying is continued until the weight of the film no longer changes. To measure the Young's modulus, traction tests are performed. The film is cut into rectangular test pieces 80 mm long and 15 mm wide. The tests are performed on a machine sold under the name Lloyd or sold under the name Zwick, under the same temperature and humidity conditions as for the drying operation, i.e., a temperature of 22±2° C. and a relative humidity of 50±5%. The test pieces are pulled at a speed of 20 mm/min and the distance between the jaws is 50±1 mm.

In another embodiment, the cosmetic process comprises applying to the hair a cosmetic composition as disclosed herein.

In one embodiment, the process comprises at least one additional operation chosen from bringing about a change in pH; an increase in temperature; adding at least one additive; and rinsing.

In one embodiment, before applying the composition as disclosed herein, a composition chosen from care, dyeing, permanent-reshaping, hair-makeup, hairstyle-fixing and hairstyle-hold compositions is applied to hair.

In the compositions as disclosed herein, the at least one polymer with a non-silicone skeleton, comprising reactive chemical functional groups, is present in a concentration ranging from 0.05% to 20% by weight, such as from 0.1% to 15% by weight and further such as from 0.25% and 10% by weight, relative to the total weight of the composition.

As disclosed herein, the composition further comprises at least one conventional cosmetic additive chosen from fixing polymers, thickeners, anionic, nonionic, cationic and amphoteric surfactants, fragrances, preserving agents, sunscreens, proteins, vitamins, provitamins, anionic, nonionic, cationic and amphoteric non-fixing polymers, mineral, plant and synthetic oils, ceramides, pseudoceramides, linear and cyclic, modified and unmodified, volatile and non-volatile silicones, pH regulators, oxidizing agents, reducing agents, inhibitors, catalysts and any other additive conventionally used in cosmetic compositions intended to be applied to the hair.

The cosmetically acceptable medium is chosen from water, at least one cosmetically acceptable solvent, for example, alcohols, esters, ketones, and cyclic volatile silicones, and water/solvent mixtures. For example, the at least one cosmetically acceptable solvent is chosen from C₁-C₄ alcohols.

When the composition as disclosed herein is packaged in an aerosol device, the composition further comprises at least one propellant, which may be chosen from volatile hydrocarbons, such as n-butane, propane, isobutane, and pentane and halogenated hydrocarbons. Carbon dioxide, nitrous oxide, dimethyl ether (DME), nitrogen or compressed air may also be used as the at least one propellant. Mixtures of propellants may also be used. For example, dimethyl ether can be used.

The at least one propellant is present, for example, in a concentration ranging from 5% to 90% by weight, and further, for example, in a concentration anging from 10% to 60% by weight, relative to the total weight of the composition in the aerosol device.

The compositions as disclosed herein may be applied to wet or dry hair.

The invention will be illustrated more fully with the aid of the following non-limiting example, wherein a.m. means active material.

### EXAMPLE

The following composition was prepared:

| | |
|---|---|
| poly(glycidyl methacrylate)⁽¹⁾ | 20 g (2% a.m.) |
| ethylenediamine | 5 g |
| aqueous ammonia containing 22% NH₃ | qs pH = 9 |
| methyl ethyl ketone | 20 g |
| water | qs 100 |

⁽¹⁾as a solution at 10% by weight in methyl ethyl ketone, sold by Polysciences, Inc.

(1) as a solution at 10% by weight in methyl ethyl ketone, sold by Polysciences, Inc.

The Young's modulus of the film obtained from the composition was about 800 MPa.

The composition was applied to the hair and when dried gave the hair a rigid coat that withstood shampooing.

What is claimed is:

1. A cosmetic composition comprising, in a cosmetically acceptable medium, at least one polymer with a non-silicone skeleton, comprising at least two non-photo-activatable reactive chemical functional groups, which may be identical or different, wherein:
(i) a film obtained by drying the composition at room temperature (22±2° C.) and at a relative humidity of 50%±5% has a Young's modulus ranging from 100 to 2000 MPa, measured at a thickness of 0.5 mm and at a traction of 20 mm/min;
(ii) the at least two non-photo-activatable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone, and α-hydroxy ketone,
lactone and thiolactone,
isocyanate,
thiocyanate,
N-hydroxysuccinimide ester,
imide,
imine,
imidate,
oxazoline, oxazolinium, oxazine, and oxazinium,
pyridylthiol,
thiosulphate,
acetoalkylate corresponding to the formula:
—OCO—A′—COCH₃,
wherein A′ is chosen from a bond, and linear and branched alkylene groups comprising from 1 to 5 carbon atoms,
AX, and
ASO₂X,
wherein:
A is a group chosen from alkylene, arylene and aralkylene groups comprising from 1 to 22 carbon atoms, which may be optionally interrupted with at least one unsaturated ring, and may optionally comprise at least one hetero atom,
X is a leaving group chosen from halogens, OSO₃H, OSO₂CH₃, OSO₂C₂H₅, OSO₂Tos, N(CH₃)₃, OPO₃R₂, and CN,
wherein Tos represents a tosylate group, and
R is chosen from a hydrogen atom and C₁ to C₅ alkyl radicals; and
(iii) the at least one polymer with a non-silicone skeleton comprising at least two reactive chemical functional groups is other than vinylbutyral/vinyl alcohol/vinyl acetate copolymer.

2. The composition according to claim 1, wherein the composition is a hair composition.

3. The composition according to claim 1, wherein the at least one hetero atom in the definition of A is chosen from N, S, and O.

4. The composition according to claim 1, wherein the polymer with a non-silicone skeleton comprising at least two reactive chemical functional groups comprises less than 50%, in numerical terms, of carboxylic acid ester functional groups, relative to the total number of reactive chemical functional groups.

5. The composition according to claim 1, wherein X is a halogen chosen from bromine, chlorine, iodine and fluorine.

6. The composition according to claim 1, wherein, in (ii), the epoxy group is monovalent and is chosen from groups corresponding to formula (I): 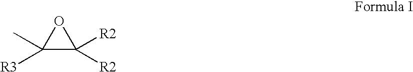
wherein R1, R2 and R3, which may be identical or different, are each chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles.

7. The composition according to claim 1, wherein, in (ii), the anhydride group is chosen from carboxylic acid anhydride groups.

8. The composition according to claim 7, wherein the carboxylic acid anhydride groups are monovalent and are chosen from groups corresponding to formula (II): 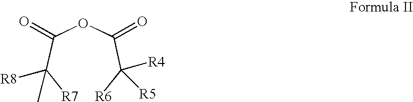
wherein R4, R5, R6, R7 and R8, which may be identical or different, are each chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles.

9. The composition according to claim 7, wherein the carboxylic acid anhydride groups are monovalent and are chosen from groups corresponding to formula (III): 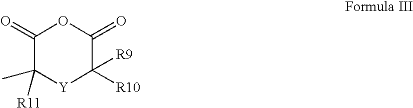
wherein Y is chosen from:
a bond,
hetero atoms chosen from O, N, S, Si and F,
alkyl and alkylene radicals that are unsubstituted or substituted with at least one radical chosen from hydroxyl and amino radicals and comprise from 1 to 5 carbon atoms,
aralkylene radicals comprising from 7 to 10 carbon atoms, and
polydimethylsiloxane radicals comprising from 1 to 6 silicon atoms; and
R9, R10 and R11, which may be identical or different, are each chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles.

10. The composition according to claim 1, wherein, in (ii), the acetoalkylate group is included in a group corresponding to formula (IV):
—R′₁—OCO—A′—COCH₃ Formula IV
wherein R′₁ is obtained by eliminating a hydrogen atom of radical R₁, and wherein the radical R1 is chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles;
and A′ is chosen from a bond, and linear and branched alkylene groups comprising from 1 to 5 carbon atoms.

11. The composition according to claim 1, wherein, in (ii), the acid chloride group is included in a group corresponding to formula (V):
—R′₁—COCl Formula V
wherein R′₁ is obtained by eliminating a hydrogen atom of radical R1, and wherein the radical R1 is chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles.

12. The composition according to claim 1, wherein, in (ii), the isocyanate group is included in a group corresponding to formula (VI):
—R′₁—NCO Formula VI
wherein R′₁ is obtained by eliminating a hydrogen atom of radical R₁, and wherein the radical R1 is chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles.

13. The composition according to claim 1, wherein, in (ii), the acetal group is monovalent and is included in at least one of the formulae (VII), (VIII) and (IX): 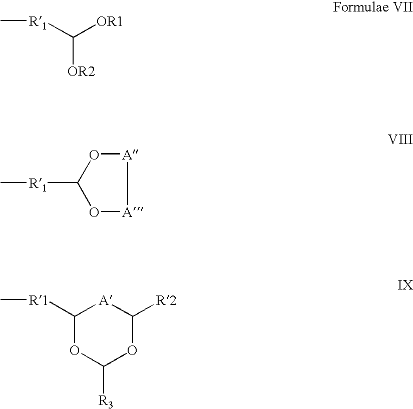
wherein:
R1, R2 and R3, which may be identical or different, are each chosen from:
a hydrogen atom;
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals;
aryl groups comprising from 6 to 22 carbon atoms;
aralkyl groups, wherein the alkyl group comprises from 1 to 20 carbon atoms; and
5- to 7-membered heterocycles;
R′₁ and R′₂ are obtained by eliminating a hydrogen atom of the radical R1 or R2;
A′ is chosen from a bond, and linear and branched alkylene groups comprising from 1 to 5 carbon atoms; and
A″ and A′″, which may be identical or different, are each chosen from linear and branched alkyl and alkylene groups comprising from 1 to 5 carbon atoms, which may be optionally interrupted with at least one hetero atom chosen from O, N, S, Si and F, and may be optionally substituted with at least one radical chosen from hydroxyl and amino radicals.

14. The composition according to claim 1, wherein the at least one polymer with a non-silicone skeleton comprising at least two reactive chemical functional groups is obtained by a process comprising at least one of the following operations:
a polycondensation;
an opening of at least one ring chosen from rings comprising from 2 to 9 carbon atoms and rings comprising from 2 to 4 silicon atoms, wherein the at least one ring may comprise at least one hetero atom; and
a polymerization of unsaturated monomers, chosen from free-radical and ionic polymerisations, by group transfer.

15. The composition according to claim 14, wherein the at least one hetero atom is chosen from N, O, S and Si.

16. The composition according to claim 1, wherein the at least one polymer with a non-silicone skeleton comprising at least two reactive chemical functional groups is present in a concentration ranging from 0.05% to 20% by weight, relative to the total weight of the composition.

17. The composition according to claim 16, wherein the at least one polymer with a non-silicone skeleton comprising at least two reactive chemical functional groups is present in a concentration ranging from 0.1% to 15% by weight, relative to the total weight of the composition.

18. The composition according to claim 17, wherein the at least one polymer with a non-silicone skeleton comprising at least two reactive chemical functional groups is present in a concentration ranging from 0.25% to 10% by weight, relative to the total weight of the composition.

19. The composition according to claim 1, further comprising at least one conventional cosmetic additive chosen from fixing polymers; thickeners; anionic, nonionic, cationic and amphoteric surfactants; fragrances; preserving agents; sunscreens; proteins; vitamins; provitamins; anionic, nonionic, cationic and amphoteric non-fixing polymers; mineral, plant and synthetic oils; ceramides; pseudoceramides; linear and cyclic, modified and unmodified, volatile and non-volatile silicones; pH regulators; oxidizing agents; reducing agents; inhibitors; and catalysts.

20. The composition according to claim 1, wherein the cosmetically acceptable medium is chosen from water, at least one cosmetically acceptable solvent, and mixtures thereof.

21. The composition according to claim 20, wherein the at least one cosmetically acceptable solvent is chosen from alcohols and cyclic volatile silicones.

22. The composition according to claim 21, wherein the alcohols are chosen from C₁-C₄ alcohols.

23. An aerosol device comprising at least one propellant and a composition comprising, in a cosmetically acceptable medium, at least one polymer with a non-silicone skeleton, comprising at least two non-photo-activatable reactive chemical functional groups, which may be identical or different, wherein:
(i) a film obtained by drying the composition at room temperature (22±2° C.) and at a relative humidity of 50%±5% has a Young's modulus ranging from 100 to 2000 MPa, measured at a thickness of 0.5 mm and at a traction of 20 mm/min;
(ii) the at least two non-photo-activatable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone, and α-hydroxy ketone,
lactone and thiolactone,
isocyanate,
thiocyanate,
N-hydroxysuccinimide ester,
imide,
imine,
imidate,
oxazoline, oxazolinium, oxazine, and oxazinium,
pyridylthiol,
thiosulphate,
acetoalkylate corresponding to the formula:
—OCO—A′—COCH₃,
wherein A′ is chosen from a bond, and linear and branched alkylene groups, comprising from 1 to 5 carbon atoms,
AX, and
ASO₂X,
wherein:
A is a group chosen from alkylene, arylene and aralkylene groups comprising from 1 to 22 carbon atoms, which may be optionally interrupted with at least one unsaturated ring, and may be optionally comprising at least one hetero atom,
X is a leaving group chosen from halogens, OSO₃H, OSO₂CH₃, OSO₂C₂H₅, OSO₂Tos, N(CH₃)₃, OPO₃R₂, and CN,
wherein Tos represents a tosylate group, and
R is chosen from a hydrogen atom and C₁ to C₅ alkyl radicals; and
(iii) the at least one polymer with a non-silicone skeleton, comprising at least two reactive chemical functional groups, is other than vinylbutyral/vinyl alcohol/vinyl acetate copolymer.

24. A process for cosmetic treatment of hair comprising applying to the hair a cosmetic composition comprising, in a cosmetically acceptable medium, at least one polymer with a non-silicone skeleton, comprising at least two non-photo-activatable reactive chemical functional groups, which may be identical or different, wherein:
(i) a film obtained by drying the composition at room temperature (22±2° C.) and at a relative humidity of 50%±5% has a Young's modulus ranging from 100 to 2000 MPa, measured at a thickness of 0.5 mm and at a traction of 20 mm/min;
(ii) the at least two non-photo-activatable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone, and α-hydroxy ketone,
lactone and thiolactone,
isocyanate,
thiocyanate,
N-hydroxysuccinimide ester,
imide,
imine,
imidate,
oxazoline, oxazolinium, oxazine, and oxazinium,
pyridylthiol,
thiosulphate,
acetoalkylate corresponding to the formula:
—OCO—A′—COCH₃,
wherein A′ is chosen from a bond, and linear and branched alkylene groups comprising from 1 to 5 carbon atoms,
AX, and
ASO₂X,
wherein:
A is a group chosen from alkylene, arylene and aralkylene groups comprising from 1 to 22 carbon atoms, which may be optionally interrupted with at least one unsaturated ring, and may be optionally comprising at least one hetero atom,
X is a leaving group chosen from halogens, OSO₃H, OSO₂CH₃, OSO₂C₂H₅, OSO₂Tos, N(CH₃)₃, OPO₃R₂, and CN,
wherein Tos represents a tosylate group, and
R is chosen from a hydrogen atom and C₁ to C₅ alkyl radicals; and
(iii) the at least one polymer with a non-silicone skeleton, comprising at least two reactive chemical functional groups, is other than vinylbutyral/vinyl alcohol/vinyl acetate copolymer.

25. The process according to claim 24, wherein before the application of the cosmetic composition, another composition chosen from care, permanent-reshaping, hair-makeup, hairstyle-fixing and hairstyle-hold compositions is applied to hair.

26. A method of forming a rigid coat on hair comprising applying to the hair a cosmetic composition comprising, in a cosmetically acceptable medium, at least one polymer with a non-silicone skeleton, comprising at least two non-photo-activatable reactive chemical functional groups, which may be identical or different, wherein:
(i) a film obtained by drying the composition at room temperature (22±2° C.) and at a relative humidity of 50%±5% has a Young's modulus ranging from 100 to 2000 MPa, measured at a thickness of 0.5 mm and at a traction of 20 mm/min;
(ii) the at least two non-photo-activatable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone, and α-hydroxy ketone,
lactone and thiolactone,
isocyanate,
thiocyanate,
N-hydroxysuccinimide ester,
imide,
imine,
imidate,
oxazoline, oxazolinium, oxazine, and oxazinium,
pyridylthiol,
thiosulphate,
acetoalkylate corresponding to the formula:
—OCO—A′—COCH₃,
wherein A′ is chosen from a bond, and linear and branched alkylene groups comprising from 1 to 5 carbon atoms,
AX, and
ASO₂X,
wherein:
A is a group chosen from alkylene, arylene and aralkylene groups comprising from 1 to 22 carbon atoms, which may be optionally interrupted with at least one unsaturated ring, and may be optionally comprising at least one hetero atom,
X is a leaving group chosen from halogens, OSO₃H, OSO₂CH₃, OSO₂C₂H₅, OSO₂Tos, N(CH₃)₃, OPO₃R₂, and CN,
wherein Tos represents a tosylate group, and
R is chosen from a hydrogen atom and C₁ to C₅ alkyl radicals; and
(iii) the at least one polymer with a non-silicone skeleton, comprising at least two reactive chemical functional groups, is other than vinylbutyral/vinyl alcohol/vinyl acetate copolymer.

27. A cosmetic composition for forming a rigid coat on hair comprising, in a cosmetically acceptable medium, at least one polymer with a non-silicone skeleton, comprising at least two non-photo-activatable reactive chemical functional groups, which may be identical or different, wherein:
(i) a film obtained by drying the composition at room temperature (22±2° C.) and at a relative humidity of 50%±5% has a Young's modulus ranging from 100 to 2000 MPa, measured at a thickness of 0.5 mm and at a traction of 20 mm/min;
(ii) the at least two non-photo-activatable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone, and α-hydroxy ketone,
lactone and thiolactone,
isocyanate,
thiocyanate,
N-hydroxysuccinimide ester,
imide,
imine,
imidate,
oxazoline, oxazolinium, oxazine, and oxazinium,
pyridylthiol,
thiosulphate,
acetoalkylate corresponding to the formula:
—OCO—A′—COCH₃,
wherein A′ is chosen from a bond, and linear and branched alkylene groups, comprising from 1 to 5 carbon atoms,
AX, and
ASO₂X,
wherein:
A is a group chosen from alkylene, arylene and aralkylene groups comprising from 1 to 22 carbon atoms, which may be optionally interrupted with at least one unsaturated ring, and may be optionally comprising at least one hetero atom,
X is a leaving group chosen from halogens, OSO₃H, OSO₂CH₃, OSO₂C₂H₅, OSO₂Tos, N(CH₃)₃, OPO₃R₂, and CN,
wherein Tos represents a tosylate group, and
R is chosen from a hydrogen atom and C₁ to C₅ alkyl radicals; and
(iii) the at least one polymer with a non-silicone skeleton, comprising at least two reactive chemical functional groups, is other than vinylbutyral/vinyl alcohol/vinyl acetate copolymer; and said composition is effective to form a rigid coat on the hair.
